SEQUENCE LISTING (4 sequences)

SEQ ID NO: 1 - protein, 35 aa, Artificial
  Synthetic General sequence of P1
  MISC_FEATURE (3)..(3): I, T or N
  MISC_FEATURE (11)..(11): D or E
  MISC_FEATURE (14)..(14): A or E
  MISC_FEATURE (19)..(19): A or K
  MISC_FEATURE (20)..(20): N or S
  MISC_FEATURE (26)..(26): D, N or S
  MISC_FEATURE (28)..(28): S or T
  MISC_FEATURE (35)..(35): R or K
SQXQQKKNEQXLLXLDKWXXLWNWFXIXNWLWYIX

SEQ ID NO: 2 - protein, 35 aa, Artificial
  Synthetic P1 clade A
SQIQQKKNEQDLLALDKWANLWNWFDISNWLWYIR

SEQ ID NO: 3 - protein, 35 aa, Artificial
  Synthetic P1 clade B
SQNQQEKNEQELLELDKWASLWNWFNITNWLWYIK

SEQ ID NO: 4 - protein, 35 aa, Artificial
  Synthetic P1 clade C
SQTQQEKNEQELLALDSWKNLWNWFSITNWLWYIK

(19) United States
(12) Patent Application Publication
(10) Pub. No.: US 2024/0016924 A1
(43) Pub. Date: Jan. 18, 2024

(54) NEW ADJUVANT TO IMPROVE THE INNATE IMMUNITY

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ PARIS CITÉ, Paris (FR)

(72) Inventors: Morgane BOMSEL, Paris (FR); Daniela TUDOR, Paris (FR)

(21) Appl. No.: 18/039,845

(22) PCT Filed: Dec. 3, 2021
(86) PCT No.: PCT/EP2021/084142

§ 371 Date: Jun. 1, 2023

(30) Foreign Application Priority Data
Dec. 4, 2020 (EP) 20306499.3

(51) Int. Cl. A61K 39/39; A61K 45/06; C07K 14/16

(52) U.S. Cl. CPC A61K 39/39; A61K 45/06; C07K 14/162; A61K 2039/55516

(57) ABSTRACT

The present invention relates to the field of adjuvant and vaccination. In the present study, the inventors investigate whether P1, in addition to being an antigen, could act as an adjuvant by first exploring its capacity to stimulate epithelial TSLP production. They evaluated additional immunomodulatory effects of P1 on human nasal mucosal models, including cytokines and chemokines production, intracellular signaling pathways, mucosal DC activation, T cell proliferation, and antigen-specific B cell responses against a model antigen in vitro. Altogether, they reported the immunological mechanism underlying P1-vaccine and the interest of P1 as a nasal mucosal adjuvant. Thus, the present invention relates to an immunoadjuvant composition comprising the P1 peptide of the HIV-1 envelope subunit gp41.

### FIELD OF THE INVENTION

The present invention relates to an immunoadjuvant composition comprising the P1 peptide of the HIV-1 envelope subunit gp41.

### BACKGROUND OF THE INVENTION

Although most human pathogens initiate infection at mucosal sites, only a few licensed mucosal vaccines have been established so far (Boyaka 2017). Intranasal immunization, by inducing an antigen-specific immunity in both the mucosal and systemic compartments, and by being applied in a atraumatic manner following pulverization, is currently considered as an ideal strategy for prevention against pathogens invading mucosa (Brandtzaeg 2011, Fujkuyama, Tokuhara et al. 2012, Zaman, Chandrudu et al. 2013). Of note, the early side effects attributed to nasal immunization, including facial nerve paralysis, are not anymore a concern as being since attributed to the specific ADP-ribosylating toxin-based adjuvant used in these studies rather than to the nasal immunization route (Jabbal-Gill 2010, Zaman, Chandrudu et al. 2013, Lycke and Lebrero-Fernandez 2018). Mucosal immunization is highly compartmentalized with unique pathways linking inducing and effector sites (Brandtzaeg 2011, Fujkuyama, Tokuhara et al. 2012). In particular, nasal vaccination elicits antigen-specific antibody responses in genital tracts (Johansson, Wassen et al. 2001) and would be therefore particularly beneficial for prevention against sexually transmitted pathogens. Nevertheless, very limited efforts have been made to understand the mechanisms by which nasal vaccines and dedicated adjuvants activate the local nasal innate and adaptive immunity as a first step to establish an effective vaccination.

P1 is a conserved 35 amino acid peptide covering the Membrane Proximal External Region (MPER) of HIV-1 envelope subunit gp41 (Alfsen and Bomsel 2002, Bomsel, Tudor et al. 2011). The MPER is a major target of broadly neutralizing antibodies and thus obviously a very interesting target for an HIV-1 vaccine. In addition, P1 mediate HIV-1 mucosal transcytosis, a principal mucosal entry pathway for HIV-1, by interacting with Galactosyl Ceramide (GalCer), the mucosal receptor of HIV-1 (Bomsel 1997, Alfsen and Bomsel 2002, Magérus-Chatinet, Yu et al. 2007). Accordingly, we have recently evaluated the protective efficacy of a gp41-subunit-virosome vaccine at mucosal sites in non-human primates (Bomsel, Tudor et al. 2011). This vaccine that used P1 as antigen linked to virosomes, an adjuvant-free vaccine carrier, was applied twice by the intramuscular route followed by two intranasal applications. In the primate model, full protection after repeated vaginal viral challenges correlated with P1/gp41-specific cervicovaginal antibodies, with IgAs blocking transcytosis and IgGs mediating antibody-dependent cell cytotoxicity (ADCC). In contrast, in protected animals, serum IgGs totally lacked antiviral activities. Furthermore, in Phase I clinical trial, we found that P1-virosome vaccination induced mucosal P1-specific antibodies with anti-viral activities (Leroux-Roels, Maes et al. 2013). These results highlighted the critical role of mucosal antibodies as the first line of defense against virus entry.

Thymic stromal lymphopoietin (TSLP) is an IL-7-like cytokine considered as a master regulator of the T helper 2 (Th2) inflammatory responses by priming dendritic cells (DCs), especially mucosal ones (Ito, Liu et al. 2012, Takai 2012). We and others recently reported that TSLP is secreted by epithelial cells during HIV-1 mucosal transmission following the interaction of the viral envelope with epithelial cells (Fontenot, He et al. 2009, Zhou, Xu et al. 2018). In turn, TSLP chemo-attract mucosal DCs to the mucosal compartment (Zhou, Xu et al. 2018), suggesting that TSLP could modulate the mucosal immune response following mucosal vaccination. Accordingly, in a study using the HIV-1 envelope gp140 as antigen, TSLP acts as a strong mucosal adjuvant in the mouse model (Van Roey, Arias et al. 2012). TSLP induced a strong humoral response both in serum and at genital level following intranasal immunization, comparable to the adjuvant effect of cholera toxin (CT) tested in parallel. In addition, a new study reported that all-trans retinoic acid shows adjuvant activity through TSLP production (Hatayama, Segawa et al. 2018). Furthermore, a recent study showed that TSLP and TSLP-receptor (TSLP-R) were up-regulated in mucosal DCs of mice nasally immunized with pneumococcal surface protein A plus CT (Joo, Fukuyama et al. 2017), and that in TSLP-R knockout mice, the specific IgA response is remarkably reduced. This indicates that TSLP and its receptor are major contributors to the mucosal adjuvant effect of CT and that TSLP-TSLP-R signaling is critical in IgA elicitation.

### SUMMARY OF THE INVENTION

In the present study, the inventors investigate whether P1, in addition to being an antigen, could act as an adjuvant by first exploring its capacity to stimulate epithelial TSLP production. They evaluated additional immunomodulatory effects of P1 on human nasal mucosal models, including cytokines and chemokines production, intracellular signaling pathways, mucosal DC activation, T cell proliferation, and antigen-specific B cell responses against a model antigen in vitro. Altogether, they reported the immunological mechanism underlying P1-vaccine and the interest of P1 as a nasal mucosal adjuvant.

Thus, the present invention relates to an immunoadjuvant composition comprising the P1 peptide of the HIV-1 envelope subunit gp41. Particularly, the invention is defined by its claims.

### DETAILED DESCRIPTION OF THE INVENTION

A first aspect of the invention relates an immunoadjuvant composition comprising the P1 peptide of the HIV-1 envelope subunit gp41.

In a particular embodiment, the immunoadjuvant composition is useful to improve the innate immunity in a subject in need thereof.

As used herein, the term “immunoadjuvant composition” refers to a composition that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “immunoadjuvant composition” means a composition that increases the cytokines and chemokines production, the intracellular signaling pathways, the mucosal DC activation, the T cell proliferation, and the antigen-specific B cell responses against an antigen.

As used herein, the term “adjuvant” refers to a substance that enhances, augments or potentiates the host's immune response to an antigen, e.g., an antigen that is part of a vaccine. Non-limiting examples of some commonly used vaccine adjuvants include insoluble aluminum compounds, calcium phosphate, liposomes, Virosomes™, ISCOMS®, microparticles (e.g., PLG), emulsions (e.g., MF59, Montanides), virus-like particles & viral vectors.

Examples of others adjuvants include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE, MF59 and RIBI (also known as SAS), which contains Monophosphoryl Lipid A (MPL) (detoxified endotoxin) from Salmonella minnesota and synthetic Trehalose Dicorynomycolate (TDM) in 2% oil (squalene)-Tween® 80-water (see for review Pulendran and Ahmed, 2011). Other examples of adjuvants include DDA (dimethyldioctadecylammonium bromide), Freund's complete and incomplete adjuvants and QuilA. In addition, immune modulating substances such as lymphokines (e.g., IFN-[gamma], IL-2 and IL-12) or synthetic IFN-[gamma] inducers such as poly I:C can be used in combination with adjuvants described herein.

The invention also relates to the P1 peptide of the HIV-1 envelope subunit gp41 for use to improve the innate immunity in a subject in need thereof.

According to the invention, the term “improve the innate immunity” or “stimulate the innate immunity” means that the innate immunity are stimulated, i.e the humoral and/or cell-mediated immune responses are stimulated/promoted. The inventors showed in the present patent application, that P1 have some immunomodulatory effects including cytokines and chemokines production, mucosal DC activation, T cell proliferation, and antigen-specific B cell responses against a model antigen in vitro.

Thus, the invention also relates to the P1 peptide of the HIV-1 envelope subunit gp41 for use as an adjuvant. Particularly, the P1 peptide can be used as a mucosal adjuvant.

The peptide P1 can also be used in the treatment of an infectious disease as adjuvant and more particularly in the treatment of an infectious disease in a subject in need thereof caused by a pathogen invading mucosa.

Thus, the invention also relates to the P1 peptide of the HIV-1 envelope subunit gp41 as an adjuvant for use in the treatment of an infectious disease in a subject in need thereof. Particularly, the P1 peptide can be used as adjuvant for use in the treatment of an infectious disease caused by a pathogen invading mucosa in a subject in need thereof.

In a particular embodiment, the invention also relates to a method for treating an infectious disease comprising administrating to a subject in need thereof a therapeutically effective amount of a P1 peptide according to the invention.

In some embodiment, the P1 peptide of the HIV-1 envelope subunit gp41 stimulates the immune response to an antigen (e.g., an antigen that is part of a vaccine).

In some embodiment, the P1 peptide of the HIV-1 envelope subunit gp41 stimulates the mucosal Dendritic cell activation, T cell proliferation, and antigen-specific B cell responses.

As used herein, the term “infectious disease” denotes all disease induced by a pathogen including virus bacteria or parasite like pneumonia, tuberculosis, COVID-19, HIV/AIDS, influenza.

As used herein the term “infectious disease caused by a pathogen invading mucosa” denotes an infectious disease caused by a pathogen which will alter the mucosa of any tissue. According to the invention, these pathogen can be a virus, a bacteria or a parasite and these diseases can be for example pneumonia, tuberculosis, COVID-19, HIV/AIDS, influenza.

In another embodiment, the invention relates i) the P1 peptide of the HIV-1 envelope subunit gp41 as an adjuvant and, ii) a treatment against an infectious diseases as a combined preparation for simultaneous, separate or sequential use in the treatment of an infectious disease in a subject in need thereof.

In a particular embodiment, the invention relates to i) the P1 peptide of the HIV-1 envelope subunit gp41 as an adjuvant and, ii) a treatment against an infectious diseases as a combined preparation for simultaneous, separate or sequential use in the treatment of an infectious disease in a subject in need thereof.

As used herein, the term “P1 peptide of the HIV-1 envelope subunit gp41” denotes the peptide P1 of the HIV-1 Glade A that is common in West Africa, the peptide P1 of the HIV-1 Glade B that predominates in Europe and the USA or the peptide P1 of the HIV-1 Glade C that predominates in Africa and China.

In one embodiment, the P1 peptide has the following general sequence (SEQ ID NO: 1): SQX₃QQKKNEQX₁₁LLX₁₄LDKWX₁₉X₂₀LWNWFX₂₆IX28NWLWYIX₃₅ wherein the amino acid X₃ is the amino acid isoleucine ((I), threonine (T) or asparagine (N), the amino acid X₁₁ is the amino acid aspartic acid (D) or glutamin acid (E), the amino acid X₁₄ is the amino acid alanine (A) or glutamin acid (E), the amino acid X₁₉ is the amino acid (alanine) A or lysine (K), the amino acid X₂₀ is the amino acid asparagine (N) or serine (S), the amino acid X₂₆ is the amino acid aspartic acid (D), asparagine (N) or serine (S) and the amino acid X₂₈ is the amino acid serine (S) or threonine (T) and the amino acid X₃₅ is the amino acid arginine (R) or lysine (K).

Particularly, the P1 peptide has one of the following sequences:

P1 clade A:
(SEQ ID NO: 2)
SQIQQKKNEQDLLALDKWANLWNWFDISNWLWYIR
P1 clade B:
(SEQ ID NO: 3)
SQNQQEKNEQELLELDKWASLWNWFNITNWLWYIK
P1 clade C:
(SEQ ID NO: 4)
SQTQQEKNEQELLALDSWKNLWNWFSITNWLWYIK

According to the invention, the P1 peptide can have an amino acid sequence having less than 60 amino acids or than less than 55 amino acids or less than 50 amino acids or less than 45 amino acids or less than 40 amino acids or less than 39 amino acids or less than 38 amino acids or less than 37 amino acids or less than 36 amino acids.

In a particular embodiment, the P1 peptide of the invention may contain one or two more amino acids at its C and N-terminal parts.

In one embodiment, the P1 peptide of to the invention comprises at least 70% identity over the P1 peptide of SEQ ID NO: 1, even more particularly at least 80%, 81%, 82%, 83%, 84%, 85%, 86%, 87%, 88%, 89%, 90%, 91%, 92%, 93%, 94%, 95%, 96%, 97%, 98%, 99% and is still able to be an adjuvant (i.e. improving the innate immunity) as described in this application.

In another particular embodiment, the P1 peptide of to the invention comprises at least 70% identity over the P1 peptide of SEQ ID NO: 2, 3 or 4, even more particularly at least 80%, 81%, 82%, 83%, 84%, 85%, 86%, 87%, 88%, 89%, 90%, 91%, 92%, 93%, 94%, 95%, 96%, 97%, 98%, 99% and is still able to be an adjuvant (i.e. improving the innate immunity) as described in this application.

In particular embodiment, the P1 peptide of the invention stimulates the immune response to an antigen (e.g., an antigen that is part of a vaccine).

In some embodiment, the P1 peptide of the invention stimulates the mucosal Dendritic cell activation, T cell proliferation, and antigen-specific B cell responses.

As used herein, the term “subject” denotes a mammal, such as a rodent, a feline, a canine, and a primate. Particularly a patient according to the invention is a human.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

A “therapeutically effective amount” as used herein is intended for a minimal amount of active agent which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient.

According to the invention, the immunoadjuvant composition can be administered in the subject in need thereof orally, parenterally (subcutaneously, intramuscularly, intravenously, intradermally or intraperitoneally), intrabuccally, intranasally, or transdermally, intralymphatically, intratumorally, intravesically, intraperitoneally and intracerebrally.

Particularly, the immunoadjuvant composition of the invention will be administrated to the subject in need thereof intranasally.

### Nucleic Acids, Vectors, Recombinant Host Cells and Uses Thereof

Another object of the invention relates to a nucleic acid sequence encoding a P1 peptide according to the invention for use to improve the innate immunity.

Another object of the invention relates to an expression vector comprising a nucleic acid sequence encoding a P1 peptide according to the invention for use to improve the innate immunity.

According to the invention, expression vectors suitable for use in the invention may comprise at least one expression control element operationally linked to the nucleic acid sequence. The expression control elements are inserted in the vector to control and regulate the expression of the nucleic acid sequence. Examples of expression control elements include, but are not limited to, lac system, operator and promoter regions of phage lambda, yeast promoters and promoters derived from polyoma, adenovirus, retrovirus, lentivirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art that the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods or commercially available.

Another object of the invention is a host cell comprising an expression vector as described here above for use to improve the innate immunity.

According to the invention, examples of host cells that may be used are eukaryote cells, such as animal, plant, insect and yeast cells and prokaryotes cells, such as E. coli. The means by which the vector carrying the gene may be introduced into the cells include, but are not limited to, microinjection, electroporation, transduction, or transfection using DEAE-dextran, lipofection, calcium phosphate or other procedures known to one skilled in the art.

In a preferred embodiment, eukaryotic expression vectors that function in eukaryotic cells are used. Examples of such vectors include, but are not limited to, viral vectors such as retrovirus, adenovirus, adeno-associated virus, herpes virus, vaccinia virus, poxvirus, poliovirus; lentivirus, bacterial expression vectors, plasmids, such as pcDNA3 or the baculovirus transfer vectors. Preferred eukaryotic cell lines include, but are not limited to, COS cells, CHO cells, HeLa cells, NIH/3T3 cells, 293 cells (ATCC #CRL1573), T2 cells, dendritic cells, or monocytes.

### Vaccine Composition and Uses Thereof

A further object of the invention relates to a vaccine composition, comprising at least one antigen, at least the P1 peptide according to the invention and optionally with one or more pharmaceutically acceptable excipients.

In one embodiment, the invention relates to a vaccine composition, comprising at least one antigen, at least the P1 peptide according to the invention and optionally with one or more pharmaceutically acceptable excipients for use to improve the innate immunity in a subject in need thereof.

In one embodiment, the invention relates to a vaccine composition, comprising at least one antigen, at least the P1 peptide according to the invention and optionally with one or more pharmaceutically acceptable excipients for use in the treatment of an infectious disease in a subject in need thereof.

A “vaccine composition”, once it has been administered to a subject or an animal, elicits a protective immune response against said one or more antigen(s) that is (are) comprised herein. Accordingly, the vaccine composition of the invention, once it has been administered to the subject or the animal, induces a protective immune response against, for example, a microorganism, or to efficaciously protect the subject or the animal against infection.

A variety of substances can be used as antigens in a compound or formulation, of immunogenic or vaccine type. For example, attenuated and inactivated viral and bacterial pathogens, purified macromolecules, polysaccharides, toxoids, recombinant antigens, organisms containing a foreign gene from a pathogen, synthetic peptides, polynucleic acids, antibodies and tumor cells can be used to prepare (i) an immunogenic composition useful to induce an immune response in a individual or (ii) a vaccine useful for treating a pathological condition.

Therefore, the immunoadjuvant composition of the invention can be combined with a wide variety of antigens to produce a vaccine composition useful for inducing an immune response in an individual and particularly for inducing an immune response against a pathogen inducing an infectious disease.

Those skilled in the art will be able to select an antigen appropriate for treating a particular pathological condition and will know how to determine whether an isolated antigen is favored in a particular vaccine formulation.

An isolated antigen can be prepared using a variety of methods well known in the art. A gene encoding any immunogenic polypeptide can be isolated and cloned, for example, in bacterial, yeast, insect, reptile or mammalian cells using recombinant methods well known in the art and described, for example in Sambrook et al., Molecular cloning: A Laboratory Manual, Cold Spring Harbor Laboratory, New York (1992) and in Ansubel et al., Current Protocols in Molecular Biology, John Wiley and Sons, Baltimore, MD (1998). A number of genes encoding surface antigens from viral, bacterial and protozoan pathogens have been successfully cloned, expressed and used as antigens for vaccine development. For example, the major surface antigen of hepatitis B virus, HbsAg, the P subunit of choleratoxin, the enterotoxin of E. coli, the circumsporozoite protein of the malaria parasite, and a glycoprotein membrane antigen from Epstein-Barr virus, as well as tumor cell antigens, have been expressed in various well known vector/host systems, purified and used in vaccines.

A pathologically aberrant cell may also be used in a vaccine composition according to the invention can be obtained from any source such as one or more individuals having a pathological condition or ex vivo or in vitro cultured cells obtained from one or more such individuals, including a specific individual to be treated with the resulting vaccine.

The vaccine composition according to the invention may contain at least one other immunoadjuvant as described above in the invention. A variety of immunoadjuvant may be suitable to alter an immune response in an individual. The type of alteration desired will determine the type of selected immunoadjuvant to be combined with the immunoadjuvant composition of the invention. For example, to enhance the innate immune response, the vaccine composition of the invention can comprise another immunoadjuvant that promotes an innate immune response, such as other PAMP or conserved region known or suspected of inducing an innate immune response. A variety of PAMPs are known to stimulate the activities of different members of the toll-like family of receptors. Such PAMPs can be combined to stimulate a particular combination of toll-like receptors that induce a beneficial cytokine profile. For example, PAMPs can be combined to stimulate a cytokine profile that induces a Th1 or Th2 immune response. Other types of immunoadjuvant that promote humoral or cell-mediated immune responses can be combined with the immunoadjuvant composition of the invention. For example, cytokines can be administered to alter the balance of Th1 and Th2 immune responses. Those skilled in the art will know how to determine the appropriate cytokines useful for obtaining a beneficial alteration in immune response for a particular pathological condition.

In another particular embodiment, the vaccine composition according to the invention, further comprises one or more components selected from the group consisting of surfactants, absorption promoters, water absorbing polymers, substances which inhibit enzymatic degradation, alcohols, organic solvents, oils, pH controlling agents, preservatives, osmotic pressure controlling agents, propellants, water and mixture thereof.

The vaccine composition according to the invention can further comprise a pharmaceutically acceptable carrier. The amount of the carrier will depend upon the amounts selected for the other ingredients, the desired concentration of the antigen, the selection of the administration route, oral or parenteral, etc. The carrier can be added to the vaccine at any convenient time. In the case of a lyophilised vaccine, the carrier can, for example, be added immediately prior to administration. Alternatively, the final product can be manufactured with the carrier.

Examples of appropriate carriers include, but are not limited to, sterile water, saline, buffers, phosphate-buffered saline, buffered sodium chloride, vegetable oils, Minimum Essential Medium (MEM), MEM with HEPES buffer, etc.

Optionally, the vaccine composition of the invention may contain conventional, secondary adjuvants in varying amounts depending on the adjuvant and the desired result. The customary amount ranges from about 0.02% to about 20% by weight, depending upon the other ingredients and desired effect. For the purpose of this invention, these adjuvants are identified herein as “secondary” merely to contrast with the above-described immunoadjuvant composition that is an essential ingredient in the vaccine composition for its effect in combination with an antigenic substance to significantly increase the humoral immune response to the antigenic substance. The secondary adjuvants are primarily included in the vaccine formulation as processing aids although certain adjuvants do possess immunologically enhancing properties to some extent and have a dual purpose.

Examples of suitable secondary adjuvants include, but are not limited to, stabilizers; emulsifiers; aluminum hydroxide; aluminum phosphate; pH adjusters such as sodium hydroxide, hydrochloric acid, etc.; surfactants such as Tween® 80 (polysorbate 80, commercially available from Sigma Chemical Co., St. Louis, Mo.); liposomes; iscom adjuvant; synthetic glycopeptides such as muramyl dipeptides; extenders such as dextran or dextran combinations, for example, with aluminum phosphate; carboxypolymethylene; bacterial cell walls such as mycobacterial cell wall extract; their derivatives such as Corynebacterium parvum; Propionibacterium acne; Mycobacterium bovis, for example, Bovine Calmette Guerin (BCG); vaccinia or animal poxvirus proteins; subviral particle adjuvants such as orbivirus; cholera toxin; N,N-dioctadecyl-N′,N′-bis(2-hydroxyethyl)-propanediamine (pyridine); monophosphoryl lipid A; dimethyldioctadecylammonium bromide (DDA, commercially available from Kodak, Rochester, N.Y.); synthetics and mixtures thereof. Desirably, aluminum hydroxide is admixed with other secondary adjuvants or an immunoadjuvant such as Quil A.

Examples of suitable stabilizers include, but are not limited to, sucrose, gelatin, peptone, digested protein extracts such as NZ-Amine or NZ-Amine AS. Examples of emulsifiers include, but are not limited to, mineral oil, vegetable oil, peanut oil and other standard, metabolizable, nontoxic oils useful for injectables or intranasal vaccines compositions.

Conventional preservatives can be added to the vaccine composition in effective amounts ranging from about 0.0001% to about 0.1% by weight. Depending on the preservative employed in the formulation, amounts below or above this range may be useful. Typical preservatives include, for example, potassium sorbate, sodium metabisulfite, phenol, methyl paraben, propyl paraben, thimerosal, etc.

The vaccine composition of the invention can be formulated as a solution or suspension together with a pharmaceutically acceptable medium.

Such a pharmaceutically acceptable medium can be, for example, water, phosphate buffered saline, normal saline or other physiologically buffered saline, or other solvent or vehicle such as glycol, glycerol, and oil such as olive oil or an injectable organic ester. A pharmaceutically acceptable medium can also contain liposomes or micelles, and can contain immunostimulating complexes prepared by mixing polypeptide or peptide antigens with detergent and a glycoside, such as Quil A.

Liquid dosage forms for oral administration of the vaccine composition of the invention include pharmaceutically-acceptable emulsions, microemulsions, solutions, suspensions, syrups and elixirs. In addition to the active ingredient(s), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the vaccine compositions of the invention for rectal or vaginal administration may be presented as a suppository, which may be prepared by mixing the active ingredient(s) with one or more suitable non-irritating excipients or carriers comprising, for example, cocoa butter, polyethylene glycol, a suppository wax or salicylate and which is solid at room temperature, but liquid at body temperature and, therefore, will melt in the rectum or vaginal cavity and release the active ingredient(s). Formulations of the present invention which are suitable for vaginal administration also include pessaries, tampons, creams, gels, pastes, foams or spray formulations containing such carriers as are known in the art to be appropriate

Vaccine compositions of this invention suitable for parenteral administration comprise the active ingredient(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and non-aqueous carriers that may be employed in the vaccine compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents that delay absorption such as aluminum monostearate and gelatin.

Injectable depot forms are made by forming microencapsule matrices of the active ingredient(s) in biodegradable polymers such as polylactide-polyglycolide. Depending on the ratio of the active ingredient(s) to polymer, and the nature of the particular polymer employed, the rate of release of the active ingredient(s) can be controlled. Examples of other biodegradable polymers include poly(orthoesters) and poly(anhydrides). Depot injectable formulations are also prepared by entrapping the active ingredient(s) in liposomes or microemulsions that are compatible with body tissue. The injectable materials can be sterilized for example, by filtration through a bacterial-retaining filter.

The formulations may be presented in unit-dose or multi-dose sealed containers, for example, ampoules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The amount of antigen and immunoadjuvant composition in the vaccine composition according to the invention are determined by techniques well known to those skilled in the pharmaceutical art, taking into consideration such factors as the particular antigen, the age, sex, weight, species, and condition of the particular animal or patient, and the route of administration.

While the dosage of the vaccine composition depends notably upon the antigen, species of the host vaccinated or to be vaccinated, etc., the dosage of a pharmacologically effective amount of the vaccine composition will usually range from about 0.01 μg to about 500 μg (and in particular 50 μg to about 500 μg) of the immunoadjuvant compound of the invention per dose.

Although the amount of the particular antigenic substance in the combination will influence the amount of the immunoadjuvant compound according to the invention, necessary to improve the immune response, it is contemplated that the practitioner can easily adjust the effective dosage amount of the immunoadjuvant compound through routine tests to meet the particular circumstances.

The vaccine composition according to the invention can be tested in a variety of preclinical toxicological and safety studies well known in the art.

For example, such a vaccine composition can be evaluated in an animal model in which the antigen has been found to be immunogenic and that can be reproducibly immunized by the same route proposed for human clinical testing.

For example, the vaccine composition according to the invention can be tested, for example, by an approach set forth by the Center for Biologics Evaluation and Research/Food and Drug Administration and National Institute of Allergy and Infectious Diseases.

Those skilled in the art will know how to determine for a particular vaccine composition, the appropriate antigen payload, route of immunization, volume of dose, purity of antigen, and vaccination regimen useful to treat a particular pathological condition in a particular animal species.

In a vaccination protocol, the vaccine may be advantageously administered as a unique dose or preferably, several times e.g., twice, three or four times at week or month intervals, according to a prime/boost mode. The appropriate dosage depends upon various parameters.

As a general rule, the vaccine composition of the present invention is conveniently administered orally, parenterally (subcutaneously, intramuscularly, intravenously, intradermally or intraperitoneally), intrabuccally, intranasally, or transdermally, intralymphatically, intratumorally, intravesically, intraperitoneally and intracerebrally. The route of administration contemplated by the present invention will depend upon the antigen.

The present invention relates to a kit comprising an immunoadjuvant composition as defined above and at least one antigen.

More particularly, the invention relates to a kit comprising:
an immunoadjuvant composition as defined above,
at least one antigen as defined above;
as combined preparation for simultaneous, separate or sequential use to induce a protective immune response against, for example, a pathogen, or to efficaciously protect the subject or the animal against infection.

The immunoadjuvant composition can be administered prior to, concomitantly with, or subsequent to the administration of at least one antigen to a subject to induce a protective immune response against, for example, a pathogen, or to efficaciously protect the subject or the animal against infection. The immunoadjuvant composition and the antigen are administered to a subject in a sequence and within a time interval such that the immunoadjuvant composition can act together with the antigen to provide an increased immune response against said antigen than if they were administered otherwise. Preferably, the immunoadjuvant composition and antigen are administered simultaneously to the subject. Also preferably, the molecules are administered simultaneously and every day to said patient.

A further aspect of the invention relates to a method for vaccinating a subject in need thereof comprising administering a pharmacologically effective amount of an antigen and a pharmacologically effective amount of an immunoadjuvant composition according to the invention.

A pharmacologically effective amount of the immunoadjuvant composition according to the invention may be given, for example orally, parenterally or otherwise, concurrently with, sequentially to or shortly after the administration of the antigen in order to potentiate, accelerate or extend the immunogenicity of the antigen.

The dosage of the vaccine composition will be dependent notably upon the selected antigen, the route of administration, species and other standard factors. It is contemplated that a person of ordinary skill in the art can easily and readily titrate the appropriate dosage for an immunogenic response for each antigen to achieve the effective immunizing amount and method of administration.

The invention will be further illustrated by the following figures and examples. However, these examples and figures should not be interpreted in any way as limiting the scope of the present invention.

### FIGURES

FIG. 1. P1 induces TSLP expression in nasal epithelial cells. (A) Confluent RPMI 2650 cells were cultured with P1 peptide (5 μM, 25 μM, 125 μM) or scramble peptide (125 μM), for 2 hours or 4 hours. TSLP secretion in culture supernatants was quantified by ELISA. (B) RPMI nasal cells were cultured with HIV-1 Glade A, B or C derived P1 peptides or the mutated P1-5W peptide at increasing concentrations for 4 hours. Inset: P1 key amino acids (661-670) corresponding to the broadly neutralizing 2F5 and 4E10 IgG epitopes with Glade-specific mutations are aligned. (C) RPMI nasal cells were pre-incubated with anti-galactosyl ceramide (GalCer) antibody for 30 min at 37° C. before stimulation with each P1 peptide. (D) Primary HNEC cells were cultured with P1 peptide (5 μM, 25 μM, 125 μM), with or without anti-GalCer pre-incubation, or P1W peptide (125 μM). Data are presented as mean±SEM (n=3-8 independent experiments; paired student's t-test *p<0.05, **p<0.01 ***p<0.001, ****p<0.001).

FIG. 2. In vitro immunization with ovalbumin adjuvanted by P1 peptide. (A): CD8-depleted PBMCs were cocultured with RPMI nasal cell monolayer for 24 hrs prior to addition of OVA as an antigen, adjuvanted by P1 at three concentrations or in the presence of mutated P1 (P1mut), in the absence of antigen or adjuvant as negative controls. CD20+ B cells expressing OVA-specific IgA or IgG were quantified by flow cytometry using anti-CD2O-PE, FITC-conjugated OVA, and APC conjugated anti-human-IgA or anti-human-IgG. Data are presented as mean±SEM (n=5 independent donors, paired student's t-test *p<0.05, “p<0.01). B and C: Mice were immunized with P1 or mutated P1 by the intra-nasal (IN) route. Resulting mucosal IgA response was evaluated in the feces after three immunizations by ELISA; unpaired student's t-test: *p<0.05 (B). Resulting OVA-specific CD8+ T cell response was evaluated by flow cytometry after staining with OVA-peptide SIINFEKL pentamers; unpaired student's t-test: **p<0.01 (C).

### EXAMPLE

Material & Methods

Peptides

Peptide P1 (aa 650-685) is derived from HIV-1 gp41 envelope subunit. P1 Glade B (SQNQQEKNEQELLELDKWASLWNWFNITNWLWYIK, SEQ ID NO: 3) was derived from the Clade B HXB2 isolate; P1 Glade A (SQIQQKKNEQDLLALD KWANLWNWFDISNWLWYIR, SEQ ID NO: 2) from the Glade A 99UGA07072 isolate, and P1-clade C (SQTQQEKNEQELLALDSWKNLWNWFSITNWLWYIK, SEQ ID NO: 4) was derived from the Clade C Bw96Bw0502 isolate. P1W is a P1 Glade B variant with W666G mutation and P1-5W with all five W mutated to G. Scramble peptide sequence comprised the same set of amino acids found in P1 Glade B but organized in a random manner (Alfsen and Bomsel 2002). Peptides were synthesized with a purity >95% by Biopeptide Co., Inc (San Diego, CA) or United BioSystems (VA, USA).

Cells

Nasal RPMI 2650 cells (isolated from the human nasal septum, squamous cell carcinoma, ATCC) were grown in MEMα (Minimum Essential Medium α, Thermo Fisher) supplemented with 10% fetal calf serum (FCS, Eurobio, Courtaboeuf, France) and 1% penicillin/streptomycin.

Primary human nasal epithelial cells (HNECs, purchased from PromoCell, Heidelberg, Germany) were isolated from nasal septum or adenoids of healthy donors. Cells from two independent donors were obtained. HNECs were cultured in airway epithelial cell basal medium (PromoCell) and supplemented with airway epithelial cell growth SupplementMix (PromoCell) and only cells from passage 2 to 6 were used.

Monocyte-derived DCs (DCs) were generated from primary human monocytes obtained from PBMCs (purity>98%) as described (Sallusto and Lanzavecchia 1994, Magérus-Chatinet, Yu et al. 2007). In brief, human peripheral blood mononuclear cells (PBMC) were separated from healthy donors blood (EFS, Paris, France), and monocytes were purified from PBMC by negative selection according to manufacturer instructions (StemCell Technologies, France). DCs were obtained by incubating monocytes for 7 days in complete medium containing GM-CSF (100 ng/ml) and IL-4 (long/ml).

Autologous CD4+ T cells were purified from PMBCs by negative selection according to manufacturer instructions (StemCell Technologies, France) (purity>95%)

Quantitative RT-PCR for TSLP

The expression of short and long form TSLP was quantified as described (Bjerkan, Schreurs et al. 2015, Dong, Hu et al. 2016). Briefly, total RNA was extracted using Trizol. Five hundred nanograms of RNA were treated with ezDNase Enzyme (Thermo Fisher) to remove genomic DNA, and reverse transcribed into cDNA using the kit SuperScript IV VILO Master Mix according to manufacturer instructions (Thermo Fisher). Quantitative PCR was performed using reported primers (Dong, Hu et al. 2016) and the PowerUp SYBR Green Master Mix according to manufacturer instructions (Thermo Fisher). Reactions were performed in triplicates, with glyceraldehyde-3-phosphate dehydrogenase (GAPDH) as the internal control. Amplification, data acquisition, and analysis were carried out using the LightCycler 480 Software (Roche, Mannheim, Germany). The levels of TSLP mRNA were normalized to the levels of GAPDH using the ΔCt method (Schmittgen and Livak 2008) and were presented as 2-ΔCt values.

MicroRNA Microarray Analysis

Confluent HNECs in 12-well plate were stimulated with medium or P1 (clade B, 125 μM) for 6 hours, at 37° C. Total RNA was extracted using Trizol. Before analysis, lfTSLP RNA up-regulation was confirmed by qPCR as described above and RNA quality was assessed with Agilent 2100 bioanalyzer according to manufacturer instructions (Agilent Technologies). Three untreated and treated paired samples from three independent experiments were analyzed by GeneChip miRNA 4.0 arrays (Affymetrix, Thermo Fisher) containing probes for 2578 human mature microRNAs and 2025 pre-mature microRNAs (https://assets.thermofisher.com/TFS-Assets/LSG/brochures/miRNA_4-0_and_4-1_datasheet.pdf). Potential microRNA targets were analyzed with the Ingenuity Pathway Analysis (IPA) software (Qiagen).

MiR-4485 Quantification and Knockdown

The quantification and knockdown of microRNA were performed as previously described with some modifications (Zhou, Xu et al. 2018). Briefly, total RNA was purified using MinElute PCR Purification Kit (Qiagen), the expression level of miR-4485 was quantified with TaqMan Small RNA Assays (Thermo Fisher). Reactions were performed in triplicates, and U6 was used as endogenous control. In order to knockdown miR-4485, 70% confluent HNEC cells were transfected with anti-miR-4485 inhibitor (67 nM, Qiagen) or mock inhibitor (miSCRIPT inhibitor negative control, 67 nM, Qiagen) using Lipofectamine RNAiMAX (Invitrogen) as described by the manufacturer. 36 h after transfection, miR-4485 expression, when quantified as described above, was reduced by 50-60% in anti-miR-4485 transfected cells as compared to anti-miR control (n=3 independent experiments).

Signaling inhibitors Confluent HNEC cells in 24-well plate were pre-incubated with inhibitors for 1 h at 37° C. prior to P1 treatment. Inhibitors, namely dexamethasone (Dex) a NF-kB and MAPK inhibitor (used at 100 nM), and Cyclosporin A (CsA) a Calcineurin Inhibitor (used at 1.5 μM), were from Invivogen and used at the manufacturer's recommended concentrations. ENMD-1068 (PAR-2 antagonist, Enzo Life Science) was used at 500 □g/mL as described (Kelso, Lockhart et al. 2006, Wygrecka, Dahal et al. 2013).

Calcium Measurement

70-80% confluent RPMI 2650 or HNEC cells in 24-well plate were loaded with 2 mM Fura-2/AM (Molecular Probes) in basal medium without serum/growth factors for 1 hour at 37° C. Cells were washed twice with mammalian saline (Conche, Boulla et al. 2009) and measurements were performed in complete medium supplied with HEPS (10 mM) and CaCl2 (2 mM) as described add (Conche, Boulla et al. 2009). Images were acquired with an inverted fluorescence microscope (Observer Z1, Zeiss, Germany) and analyzed with MetaMorph software (Guichard, Bonilla et al. 2017) Calcium was measured every 5 seconds by video fluorescence imaging. Results were expressed as 340 nm to 380 nm fluorescence ratio and normalized to the baseline, i.e. ratio at time zero was set as 1.

Cytokines and Chemokines Quantification

TSLP, IL-25/IL-17E, IL-33, IFN-γ, IL-10, IL-12/23p40, IL-4, IL-5, IL-6, IL-13, TNF-α, MMP-9, IL-8/CXCL8, MIP-3α/CCL2, MCP-1/CCL20, MDC/CCL22, TARC/CCL17, APRIL and BAFF were measured in culture supernatants from indicated experiments with custom multiplex Luminex assays (Bio-techne) according to the manufacturer's instructions. Additionally, when indicated TSLP was measured in culture supernatants by enzyme-linked immunosorbent assay (ELISA) with a limit of detection of 8 pg/ml (Thermo Fisher) according to the manufacturer's instructions.

DC-EC Co-Culture and DC Activation

Three DC culture systems were developed. Monocytes derived DCs (5×105 cells) were incubate for 24 h in medium alone and considered as non-mucosal DCs (DCs) or co-cultured with nasal epithelial cell (RPMI-2650 cell line) monolayer in 24-well plate (DC-EC or eduDC systems for 24 hours at 37° C. In turn, DCs were either further cultured with ECs during P1 stimulation (DC-ECs) or separated from EC and transferred into a new plate (eduDCs) for further P1 treatment. Subsequently, P1 (clade B, 125 μM) or medium were added to each of the DCs, DC-ECs or eduDCs cultures for 16 hrs. DCs were collected for surface staining with allophycocyanin (APC)-conjugated anti-CD86, R-phycoerythrin (PE)-conjugated anti-CD83, APC-conjugated anti-TSLPR, PE-conjugated anti-IL-7Rα antibodies (all from Bio-Techne). Specific labeling was quantified by flow cytometry using a Guava EasyCyte flow cytometer and the InCyte software (Merck) described (Duchemin, Khamassi et al. 2018). Culture supernatant were collected and frozen at −80° C. for subsequent cytokine and chemokine analyses.

DC-T Co-Cultures

DCs and confluent ECs were co-cultured overnight as described above, and DC-EC or eduDC was further incubated with P1 (clade B, 125 μM) or medium for 24 h. Then, DCs were separated and incubated with autologous CD4+ T cells pre-labeled with CFSE (Thermo Fisher) according to the manufacturer's instructions, at a ratio of 1:5 (DC/T). After 5 days of culture, CD4+ T cell proliferation was analyzed by flow cytometry as described (Yeh, Yeh et al. 2013, Qin, Yin et al. 2015) using Phytohaemagglutinin (PHA) (5 μg/mL) as positive control.

In Vitro Immunization Assay

In vitro immunization assay was performed as reported (Jung, Matsumoto et al. 2007) with modifications. Briefly, 1×106 CD8-depleted PBMCs (Human CD8 Depletion Cocktail, StemCell Technologies, France) were cocultured for 24 hrs with RPMI 2650 cells (1×105) pre-seeded in 48 well plates for 48 hrs. Then, ovalbumin (OVA, EndoFit Ovalbumin, 10 □g/mL, Invivogen) alone, OVA together with P1 (5 μM, 25 μM, 125 μM), OVA together with P1 mutant (P1mut, 125 μM), or medium were added to in RPMI 1640 medium supplemented with Non-Essential Amino Acids (NEAA solution, Thermo Fisher), IL-4 (long/mL), IL-2 (10 UI/mL) and 2-mercaptoethanol (20 μM) for 7 days.

For the detection of OVA-specific B cells, at indicated time of culture, PMBCs were surface stained with ovalbumin conjugated to fluorescein (OVA-FITC, 20 ug/mL, Thermo), PE-conjugated mouse anti-human CD20 (BD Biosciences, CA, USA), APC-conjugated goat anti-human IgA or donkey anti-human IgG (Jackson ImmunoResearch, PA, USA) as indicated by the manufacturer. Specific labeling was quantified by flow cytometry with a Guava EasyCyte flow cytometer (Merck-Millipore), and analyzed with the dedicated InCyte software, using the following strategy: CD20+ B cells were first gated and cell double positive for OVA-FITC+ and APC-conjugated anti-IgA or anti-IgG were determined as OVA-IgA or IgG-specific B-cell s.

Statistical Analysis.

Data are presented as mean□SEM of at least three independent experiments. Statistical significance was analyzed by the two-tailed Student's t-test with the GraphPad Prism software.

Results

P1 Induces TSLP Secretion in Nasal Epithelial Cells by Interacting with Galactosyl Ceramide.

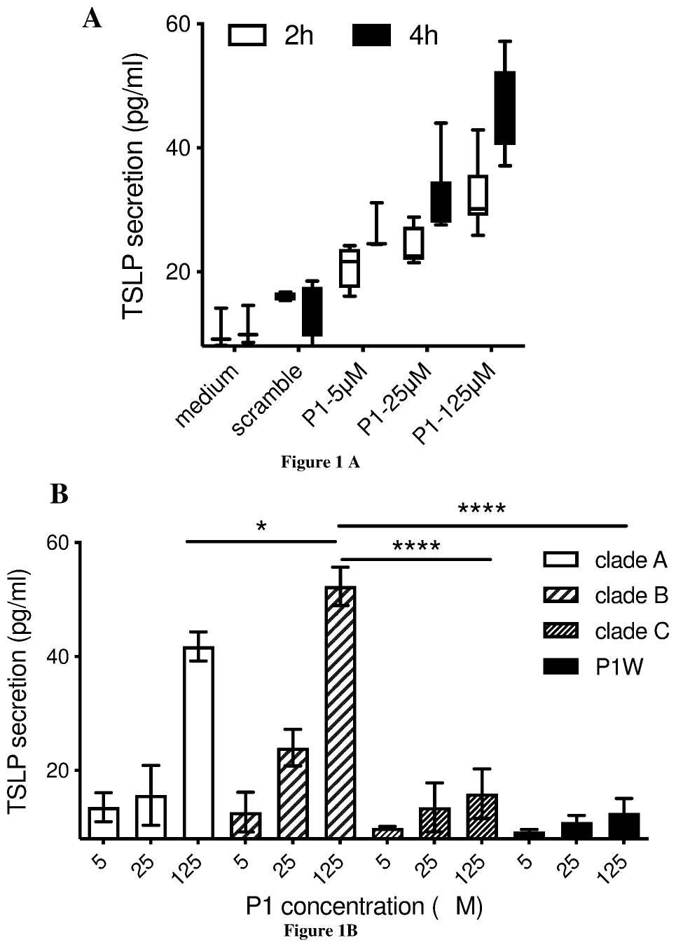

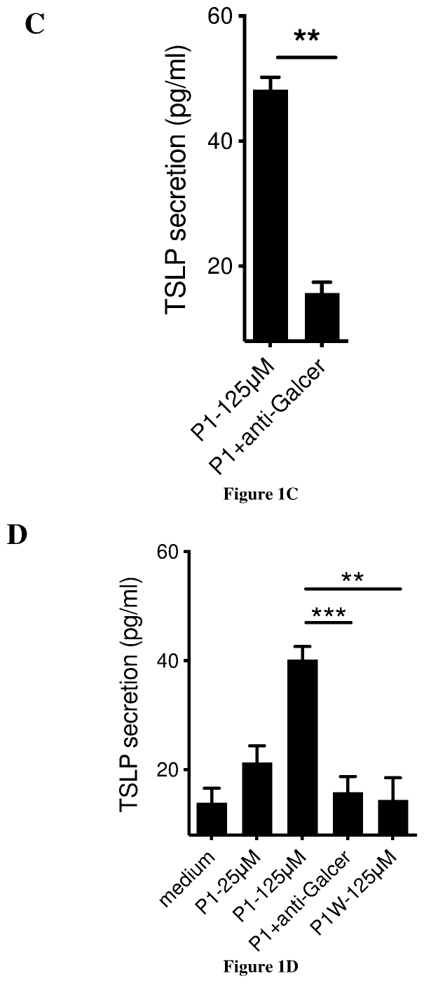

We first investigate whether P1 induced TSLP secretion in nasal epithelium. Therefore, we cultured human nasal epithelial cells (RPMI 2650) with P1 Glade B for 2-24 hours at 37° C. and analyzed the culture supernatants for TSLP secretion. Compared with the medium and scramble peptides used as negative controls, P1 upregulates TSLP secretion in a dose-dependent manner from 2 h to 4 h (FIG. 1A). At 125 μM, when P1 adopts a trimeric oligomerization state (Alfsen and Bomsel 2002), P1 induces a significantly higher secretion of TSLP than in a monomeric state (at 5 μM, and 25 μM). TSLP secretion occurs rapidly within hours post stimulation reaching a plateau from 4 to 24 h.

Although P1 sequence is relatively conserved, in contrast to highly mutated regions of HIV-1 envelope gp120, P1 sequence varies between HIV-1 Glade A that is common in West Africa, Glade B that predominates in Europe and the USA, and Glade C that predominates in Africa and China (FIG. 1B). Consequently, we next analyzed whether TSLP secretion was restricted to Glade B derived P1, or would also be stimulated by P1 derived from Glade A and C viruses (FIG. 1B). Secretion of TSLP induced by Clade A compared to Glade B P1 is reduced by 20% (41.8±2.6 pg/mL for Glade A, 52.3±3.4 pg/mL for Clade B P1 at 125 uM, p<0.05, n=5) whereas P1 Glade C failed to induce TSLP secretion. P1 Glade C differs from P1 Glade B and A by the ELDKW motif, we have previously shown to be determinant in P1 Glade B binding to Galactosyl Ceramide (GalCer), the epithelial HIV-1 receptor (Bomsel 1997, Alfsen and Bomsel 2002). We thus hypothesized that P1 Glade B and A interaction with GalCer initiated TSLP secretion. Accordingly, P1 Glade B mutated in W666G (P1W) that fails to interact with GalCer (Alfsen and Bomsel 2002) completely looses the capacity to induce TSLP secretion. Furthermore, when the interaction between P1 and GalCer was blocked by pre-incubation with anti-GalCer antibody, TSLP production is entirely blocked, confirming that TSLP secretion is initiated by P1 interaction with GalCer (FIG. 1C). Importantly, P1 stimulation also induces primary human nasal epithelial cells (HNECs) to secrete TSLP upon a GalCer-dependent manner (FIG. 1D).

Long Form TSLP is Up-Regulated after P1 Stimulation

Two transcript variants of TSLP, namely the short (sfTSLP) and the long (lfTSLP) forms, were recently identified (Tsilingiri, Fornasa et al. 2017). The expression of sfTSLP has been suggested to be constitutive and homeostatic whereas the lfTSLP leads to proinflammatory responses (Tsilingiri, Fornasa et al. 2017). We thus investigated which form(s) of TSLP was upregulated by P1 stimulation of nasal epithelial cells. When analyzed at the transcriptional level in nasal RPMI and primary HNEC cells, the expression of sfTSLP and lfTSLP differs by a factor >102. Upon P1 stimulation of both nasal RPMI cells and primary HNECs, the level of the sfTSLP transcript remains unchanged (data not shown). In contrast upon P1 stimulation, the level of lfTSLP transcription in both nasal RPMI cells and HNECs increased by 1.9 (p=0.02, n=5) and 5.9-fold (p=0.004, n=6), respectively, compared to unstimulated cells. Altogether, these results indicate that P1 upregulates lfTSLP selectively at a transcription level.

P1-Induced lfTSLP Expression is Regulated by miR-4485, Calcineurin and PAR-2

Next, we investigated the intracellular mechanisms leading to TSLP expression after P1 interaction with GalCer. We concentrated on primary HNECs as its increase in lfTSLP transcription level upon P1 stimulation is higher compared to that in nasal RPMI cells (data not shown).

We have previously shown that the non-coding microRNA miR-375 controls TSLP expression in primary human foreskin keratinocytes (Zhou, Xu et al. 2018), as it does in human intestinal cell lines (Biton, Levin et al. 2011). When tested in primary HNECs, we found that the TSLP secretion induced by P1 described above is not accompanied by a change in miR-375 expression. We thus further investigated the microRNAs profiles upon nasal epithelial HNECs stimulation by P1 after treatment with or in absence of P1 for 6 hours, comparatively by microRNA array analysis. As a result, 39 microRNAs are differentially expressed with a fold change ranging from 1.3 to 9.15 when p<0.05, including 23 up-regulated and 16 down-regulated genes (data not shown).

Remarkably, in the microRNA array analysis, the highest upregulated gene upon P1 stimulation is the miR-4485-3p with a >9-fold increase (data not shown). We validated this up-regulation by qPCR resulting in an increase in miR-4485-3p expression by 2.6±0.8-fold (n=4) upon P1 stimulation (data not shown). MiR-4485-3p is a relatively newly described microRNA that is poorly characterized at the experimental level. The only described activity of miR-4485-3p is to regulate mitochondrial functions suggesting a role in tumor suppression (Sripada, Singh et al. 2017). Thus, we first evaluated whether this microRNA controlled TSLP expression. Therefore, primary HNECs were transfected with a specific siRNA to inhibit miR-4485-3p expression before P1 stimulation. As a result, knocking down miR-4485-3p by 50-60% decreases, in turn, P1-induced TSLP expression by 48±10% (p<0.01, n=4), compared to cells transfected with a mock inhibitor (data not shown).

Bioinformatics analyses were conducted to further elucidate the mechanisms by which P1 modulates all identified microRNAs and subsequent intracellular signaling pathways. The genes predicted to be targeted by identified microRNAs participate in several signaling pathways, the five principals including G protein-coupled receptor (GPCR) associated signaling, Nuclear factor of activated T-cells (NFAT) signaling, Rho GDP signaling, Ephrin receptor signaling, and thrombin signaling (data not shown).

Corroborating this predictive analysis designating NFAT, GPCR, and thrombin (PAR associated) pathways (Coughlin 2000) as the main ones induced by P1 stimulation, it has been described that in keratinocytes, TSLP production is regulated by Ca2+-dependent NFAT signaling itself triggered by the activation of GPCR protease-activated receptor 2 (PAR-2) (Wilson, The et al. 2013). Thus, we next evaluated experimentally whether inhibitors specific to these pathways also reduced P1-induced TSLP expression in primary HNECs. Therefore, HNECs were pre-treated with the calcineurin inhibitor Cyclosporine A (CsA), or with the PAR-2 antagonist ENMD-1068 prior to P1 stimulation. Accordingly, TSLP expression was reduced by 67±4% (p<0.001, n=3) upon CsA pre-treatment and by 46±24% (p<0.05, n=3) following ENMD-1068 pre-treatment (data not shown). Furthermore, CsA and ENMD-1068 inhibitors also blocked the up-regulation of miR-4485-3p (data not shown). In contrast, blocking NF-κB and MAPK with Dexamethasone (Dex) had no effect on TSLP expression (data not shown). These results provide direct and indirect evidence that miR-4485-3p, calcineurin, and PAR-2 mediated signaling tightly correlate with P1-induced TSLP expression.

To further confirm that P1 activates calcineurin, we investigated whether, in nasal epithelial cells, P1 induces calcium fluxes that generally causes calcineurin activation (Hogan, Chen et al. 2003). Accordingly, using fluorescent dye Fura-2/AM imaging technology, we observed in both nasal RPMI cells and primary HNEC cells, that P1 treatment induces an immediate extracellular calcium influx in a concentration-dependent manner (125 μM vs 2511M of P1, n=3) (data not shown). In contrast, treatment with control peptides (P1-5W mutant and P1 Glade C, both at 125 μM) fails to raise the calcium level significantly.

Together, these data indicated that in nasal epithelial cells, P1-stimulated TSLP expression is regulated by miR-4485 via a Ca2+-dependent NFAT signaling pathway through the interaction with PAR-2 receptor.

P1 Further Stimulates Epithelial Secretion of MMP-9, CCL20, CCL2, and IL-10

We next investigated whether, in addition to TSLP, P1 could stimulate epithelial secretion of additional immune factors prone to attract antigen presenting cells (APCs). Therefore, nasal RPMI cells where incubated with P1 (125 μM) and after 24 hrs, the cell culture medium was analyzed for interleukin (IL)-25/IL-17E, IL-33, IFN-γ, IL-10, IL-12/23p40, IL-4, IL-5, IL-6, IL-13, TNF-α, Matrix metalloproteinase 9 ((MMP-9), IL-8/CXCL8, MIP-3α/CCL2, MCP-1/CCL20, MDC/CCL22, TARC/CCL17, APRIL and BAFF by Luminex technology. As a result, P1 selectively induced the secretion of MMP-9, CCL20, CCL2 and IL-10 (data not shown). Furthermore, as observed for P1-induced TSLP secretion, P1W and P1 Glade C were unable to stimulate significant MMP-9, CCL-20 CCL2 or IL-10 production. Together with TSLP (Zhou, Xu et al. 2018), this set of immune factors could facilitate recruitment of APCs to the mucosal surface for initiation of an immune response, since CCL20 and CCL2 chemo-attract macrophages and immature dendritic cells (DCs), and MMP-9 degrades the extracellular matrix and facilitates the migration of immune cells in or out the epithelium. Treg cells have IgA-inducing functions and require RA, TGF-b1, IL-10, and TSLP from intestinal epithelial cells and DCs. So, we assumed IL-10 released from either EC or DC may contribute to IgA class switching (Gutzeit, Magri, et al. 2014).

P1 Activates Human Dendritic Cells in a Nasal Mucosal Model

APCs link the innate and adaptive immune systems and determine the polarization of the immune responses. APCs are thus a key target in vaccine and adjuvant development (Coffman, Sher et al. 2010). DCs being the most abundant APCs in airway mucosa (Schon-Hegrad, Oliver et al. 1991), we further investigated mucosal DCs responses to P1 stimulation.

It has been suggested that mucosal DCs display unique functions due to the local microenvironment, especially at mucosal level (Brandtzaeg 2009). In particular, mucosal DCs modulate their functions by interacting with epithelial cells (ECs) including via epithelial secretion of TSLP (Rimoldi, Chieppa et al. 2005, Biton, Levin et al. 2011). Thus, we established a simplified mucosal DC model, by co-culturing DCs and nasal ECs (RPMI-2650 cell line), thereby mimicking the nasal mucosal environment. DCs were first ‘educated’ by a 24 hr co-culture with ECs. Subsequently, these ‘educated’ DCs were either maintained in culture with ECs and referred to as DC-EC, or separated from the epithelium and referred to as eduDC. Alternatively, DCs only cultured with medium represented ‘non-mucosal’ DCs.

Each type of DCs was stimulated with P1 overnight and the expression of maturation markers was assessed by flow cytometry. Compared to untreated cells, P1-treated mucosal DCs, either DC-EC or eduDCs, show a significant up-regulation of co-stimulatory molecules CD83 (data not shown) and CD86 (data not shown). In contrast, P1 has no effect on ‘non-mucosal’ DCs. Surprisingly, P1 also significantly enhanced the expression of TSLP receptor, with both chain TSLP-R (data not shown) and IL-7Rα (data not shown) being upregulated on the DCs in all three models.

The cytokine and chemokine secretion profile were also studied in these models, comparatively. Compared with non-mucosal DCs, P1 induces a significant increase in IL-6, IL-8, IL-10, CCL20, CCL22 and MMP-9 secretion in eduDC and DC-EC models as well as that of TSLP secretion, although more modest. In contrast, IFN-γ secretion remains unchanged upon P1-stimulation or slightly decreases in DC-EC (data not shown). In addition, several cytokines, such as IL-25, IL-33, IL-4, IL-5, remain undetectable whatever the model, whereas others, such as IL-12, IL-13, CCL2, CCL17, TNF-α, APRIL and BAFF, are secreted equally in all three models.

Activated DCs are known to stimulate T-cell proliferation to initiate an adaptive immune response, both in vivo and in vitro (Fontenot, He et al. 2009, Yeh, Yeh et al. 2013, Qin, Yin et al. 2015). Therefore, we further assessed if P1 activated mucosal DCs could promote T cell proliferation. As a result, P1 primed eduDCs induced the proliferation of autologous CD4+ T cells, whereas treatment with control peptides (P1W mutant and P1 Glade C) or P1 stimulation on CD4+ T cells alone has no effect (data not shown). Similar results were observed with DC-ECs, in agreement with the similar cytokine profiles between DC-EC and eduDC as described above.

Altogether, these results show that P1 activates mucosal DCs specifically, resulting in Th2 cytokine and chemokine secretion, and in CD4+ T cell proliferation.

P1 Acts as an Adjuvant to Stimulate Antigen-Specific Humoral Responses In Vitro

Finally, given that P1 induces various immuno-modulatory effect in mucosal cells involved in vaccination at the nasal site, as described above, we investigated whether P1 was able to act as an adjuvant. Using an in vitro immunization model with human PBMCs, the capacity of P1 to trigger a humoral immune response against a well-characterized antigen, namely ovalbumin (OVA), was evaluated.

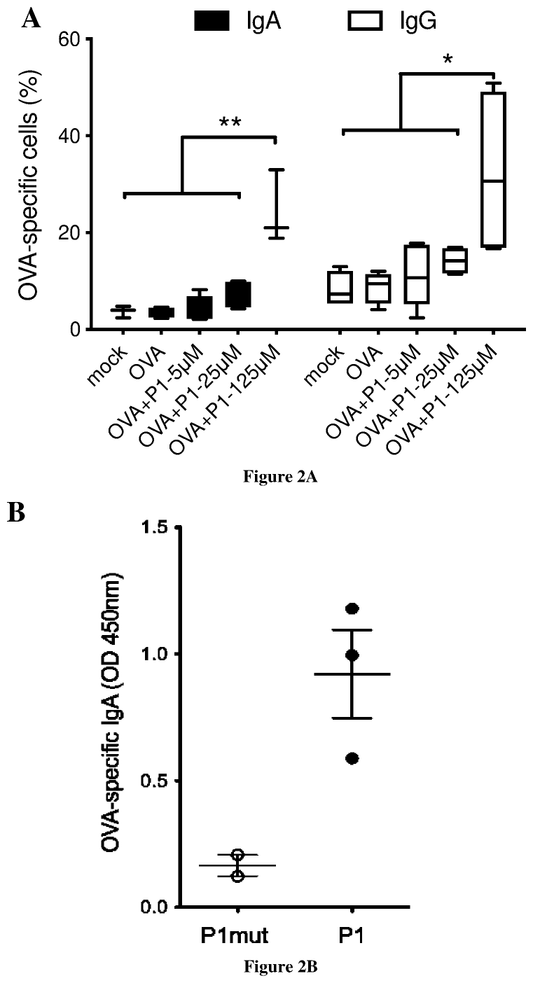

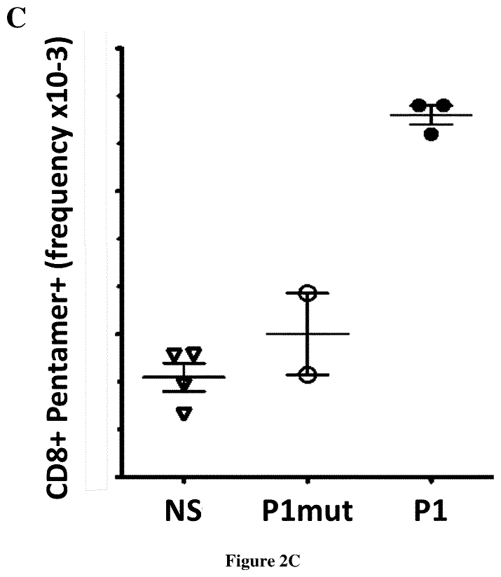

In vitro immunization assays have been used to produce specific monoclonal antibodies using a defined antigen complemented with an adjuvant (Borrebaeck, Danielsson et al. 1987). Here, we establish a mucosal immunization model adapted from (Jung, Matsumoto et al. 2007, Yeh, Yeh et al. 2013, Wijkhuisen, Savatier et al. 2016) and using mucosal DCs based on our results presented above. OVA was selected as the model antigen. Therefore, human CD8-depleted PBMCs (n=5 independent donors) were cocultured with RPMI 2650 cells for one day to educate DCs, and prior to addition of either medium, OVA, OVA plus P1 mutant (P1mut, 125 μM) or OVA plus P1 (5 μM, 25 μM, 125 μM) for seven more days. OVA-specific B cells were quantified by flow cytometry using FITC-conjugated OVA and anti-CD2O-PE to gate on OVA-specific B cells. The Ig isotype of surface B cell receptor (BCR) was next characterized by APC-conjugated anti-human-IgA or anti-human-IgG. As shown in FIG. 2 (A, B and C), OVA alone, similarly to medium, failed to induce OVA-specific specific B cells, whereas in the presence of P1, OVA-specific B cells were detected. At 5 μM and 25 μM, the concentration at which P1 remains in the monomeric state, induction of OVA-specific B cells is very limited, whereas, at 125 μM, P1 significantly enhances OVA recognition by B cells due to surface expression of OVA-specific IgA and IgG isotypes. Similar results were obtained when B cells were stained with anti-CD19-PE. Importantly, in the absence of nasal epithelial cells during the in vitro immunization, P1 is not able to induce OVA-specific B cells. Within the culture supernatants of day 7, OVA-specific antibody secretion could not be detected by ELISA, most likely because blasts were not formed at this early time point of the immunization and in agreement with the detection of OVA-specific IgA and IgG at the B cell surface, prior to blast differentiation. Hence, P1 appears to act as an adjuvant by promoting the expression of antigen-specific BCR on B cells, which may need additional signals to develop into plasma cells.

### REFERENCES

Throughout this application, various references describe the state of the art to which this invention pertains. The disclosures of these references are hereby incorporated by reference into the present disclosure.
Alfsen, A. and M. Bomsel (2002). “HIV-1 gp41 envelope residues 650-685 exposed on native virus act as a lectin to bind epithelial cell galactosyl ceramide.” J Biol Chem 277(28): 25649-25659.
Anjuere, F., A. George-Chandy, F. Audant, D. Rousseau, J. Holmgren and C. Czerkinsky (2003). “Transcutaneous immunization with cholera toxin B subunit adjuvant suppresses IgE antibody responses via selective induction of Th1 immune responses.” J Immunol 170(3): 1586-1592.
Biton, M., A. Levin, M. Slyper, I. Alkalay, E. Horwitz, H. Mor, S. Kredo-Russo, T. Avnit-Sagi, G. Cojocaru, F. Zreik, Z. Bentwich, M. N. Poy, D. Artis, M. D. Walker, E. Hornstein, E. Pikarsky and Y. Ben-Neriah (2011). “Epithelial microRNAs regulate gut mucosal immunity via epithelium-T cell crosstalk.” Nat Immunol 12(3): 239-246.
Bjerkan, L., O. Schreurs, S. A. Engen, F. L. Jahnsen, E. S. Baekkevold, I. J. Blix and K. Schenck (2015). “The short form of TSLP is constitutively translated in human keratinocytes and has characteristics of an antimicrobial peptide.” Mucosal Immunol 8(1): 49-56.
Bomsel, M. (1997). “Transcytosis of infectious human immunodeficiency virus across a tight human epithelial cell line barrier.” Nat Med 3(1): 42-47.
Bomsel, M., D. Tudor, A. S. Drillet, A. Alfsen, Y. Ganor, M. G. Roger, N. Mouz, M. Amacker, A. Chalifour, L. Diomede, G. Devillier, Z. Cong, Q. Wei, H. Gao, C. Qin, G. B. Yang, R. Zurbriggen, L. Lopalco and S. Fleury (2011). “Immunization with HIV-1 gp41 subunit virosomes induces mucosal antibodies protecting nonhuman primates against vaginal SHIV challenges.” Immunity 34(2): 269-280.
Borrebaeck, C. A., L. Danielsson and S. A. Moller (1987). “Human monoclonal antibodies produced from L-leucine methyl ester-treated and in vitro immunized peripheral blood lymphocytes.” Biochem Biophys Res Commun 148(3): 941-946.
Boyaka, P. N. (2017). “Inducing Mucosal IgA: A Challenge for Vaccine Adjuvants and Delivery Systems.” J Immunol 199(1): 9-16.
Brandtzaeg, P. (2009). “Mucosal immunity: induction, dissemination, and effector functions.” Scand J Immunol 70(6): 505-515.
Brandtzaeg, P. (2011). “Potential of nasopharynx-associated lymphoid tissue for vaccine responses in the airways.” Am J Respir Crit Care Med 183(12): 1595-1604.
Coffman, R. L., A. Sher and R. A. Seder (2010). “Vaccine adjuvants: putting innate immunity to work.” Immunity 33(4): 492-503.
Conche, C., G. Boulla, A. Trautmann and C. Randriamampita (2009). “T cell adhesion primes antigen receptor-induced calcium responses through a transient rise in adenosine 3′,5′-cyclic monophosphate.” Immunity 30(1): 33-43.
Coughlin, S. R. (2000). “Thrombin signalling and protease-activated receptors.” Nature 407: 258.
Dong, H., Y. Hu, L. Liu, M. Zou, C. Huang, L. Luo, C. Yu, X. Wan, H. Zhao, J. Chen, Z. Xie, Y. Le, F. Zou and S. Cai (2016). “Distinct roles of short and long thymic stromal lymphopoietin isoforms in house dust mite-induced asthmatic airway epithelial barrier disruption.” Sci Rep 6: 39559.
Duchemin, M., M. Khamassi, L. Xu, D. Tudor and M. Bomsel (2018). “IgA Targeting Human Immunodeficiency Virus-1 Envelope gp41 Triggers Antibody-Dependent Cellular Cytotoxicity Cross-Clade and Cooperates with gp41-Specific IgG to Increase Cell Lysis.” Frontiers in Immunology 9(244).
Fontenot, D., H. He, S. Hanabuchi, P. N. Nehete, M. Zhang, M. Chang, B. Nehete, Y. H. Wang, Y. H. Wang, Z. M. Ma, H. C. Lee, S. F. Ziegler, A. N. Courtney, C. J. Miller, S. C. Sun, Y. J. Liu and K. J. Sastry (2009). “TSLP production by epithelial cells exposed to immunodeficiency virus triggers DC-mediated mucosal infection of CD4+ T cells.” Proc Natl Acad Sci USA 106(39): 16776-16781.
Fujkuyama, Y., D. Tokuhara, K. Kataoka, R. S. Gilbert, J. R. McGhee, Y. Yuki, H. Kiyono and K. Fujihashi (2012). “Novel vaccine development strategies for inducing mucosal immunity.” Expert Rev Vaccines 11(3): 367-379.
George, J. A., S. B. Kim, J. Y. Choi, A. M. Patil, F. M. A. Hossain, E. Uyangaa, J. Hur, S. Y. Park, J. H. Lee, K. Kim and S. K. Eo (2017). “TLR2/MyD88 pathway-dependent regulation of dendritic cells by dengue virus promotes antibody-dependent enhancement via Th2-biased immunity.” Oncotarget 8(62): 106050-106070.
Guichard, V., N. Bonilla, A. Durand, A. Audemard-Verger, T. Guilbert, B. Martin, B. Lucas and C. Auffray (2017). “Calcium-mediated shaping of naive CD4 T-cell phenotype and function.” Elife 6.
Gutzeit, C., Magri, G., and Cerutti, A. (2014). “Intestinal IgA production and its role in host-microbe interaction.” Immunol Rev. Jul; 260(1): 76-85.
Hatayama, T., R. Segawa, N. Mizuno, S. Eguchi, H. Akamatsu, M. Fukuda, F. Nakata, W. J. Leonard, M. Hiratsuka and N. Hirasawa (2018). “All-Trans Retinoic Acid Enhances Antibody Production by Inducing the Expression of Thymic Stromal Lymphopoietin Protein.” J Immunol 200(8): 2670-2676.
Hogan, P. G., L. Chen, J. Nardone and A. Rao (2003). “Transcriptional regulation by calcium, calcineurin, and NFAT.” Genes Dev 17(18): 2205-2232.
Ito, T., Y.-J. Liu and K. Arima (2012). “Cellular and Molecular Mechanisms of TSLP Function in Human Allergic Disorders—TSLP Programs the “Th2 code” in Dendritic Cells.” Allergology International 61(1): 35-43.
Jabbal-Gill, I. (2010). “Nasal vaccine innovation.” J Drug Target 18(10): 771-786.
Johansson, E. L., L. Wassen, J. Holmgren, M. Jertborn and A. Rudin (2001). “Nasal and vaginal vaccinations have differential effects on antibody responses in vaginal and cervical secretions in humans.” Infect Immun 69(12): 7481-7486.
Joo, S., Y. Fukuyama, E. J. Park, Y. Yuki, Y. Kurashima, R. Ouchida, S. F. Ziegler and H. Kiyono (2017). “Critical role of TSLP-responsive mucosal dendritic cells in the induction of nasal antigen-specific IgA response.” Mucosal Immunol 10(4): 901-911.
Jung, Y. S., S. E. Matsumoto, M. Yamashita, K. Tomimatsu, K. Teruya, Y. Katakura and S. Shirahata (2007). “Propionibacterium acnes acts as an adjuvant in in vitro immunization of human peripheral blood mononuclear cells.” Biosci Biotechnol Biochem 71(8): 1963-1969.
Kamekura, R., T. Kojima, J. Koizumi, N. Ogasawara, M. Kurose, M. Go, A. Harimaya, M. Murata, S. Tanaka, H. Chiba, T. Himi and N. Sawada (2009). “Thymic stromal lymphopoietin enhances tight-junction barrier function of human nasal epithelial cells.” Cell Tissue Res 338(2): 283-293.
Kelso, E. B., J. C. Lockhart, T. Hembrough, L. Dunning, R. Plevin, M. D. Hollenberg, C. P. Sommerhoff, J. S. McLean and W. R. Ferrell (2006). “Therapeutic promise of proteinase-activated receptor-2 antagonism in joint inflammation.” J Pharmacol Exp Ther 316(3): 1017-1024.
Kurt-Jones, E. A., L. Popova, L. Kwinn, L. M. Haynes, L. P. Jones, R. A. Tripp, E. E. Walsh, M. W. Freeman, D. T. Golenbock, L. J. Anderson and R. W. Finberg (2000). “Pattern recognition receptors TLR4 and CD14 mediate response to respiratory syncytial virus.” Nat Immunol 1(5): 398-401.
Leroux-Roels, G., C. Maes, F. Clement, F. van Engelenburg, M. van den Dobbelsteen, M. Adler, M. Amacker, L. Lopalco, M. Bomsel, A. Chalifour and S. Fleury (2013). “Randomized Phase I: Safety, Immunogenicity and Mucosal Antiviral Activity in Young Healthy Women Vaccinated with HIV-1 Gp41 P1 Peptide on Virosomes.” PLoS One 8(2): e55438.
Lycke, N. and C. Lebrero-Fernandez (2018). “ADP-ribosylating enterotoxins as vaccine adjuvants.” Curr Opin Pharmacol 41: 42-51.
Magérus-Chatinet, A., H. Yu, S. Garcia, E. Ducloux, B. Terris and M. Bomsel (2007). “Galactosyl ceramide expressed on dendritic cells can mediate HIV-1 transfer from monocyte derived dendritic cells to autologous T cells.” Virology 362(1): 67-74.
Meng, H., H. Li, R. Ohe, Y. A. Naing, S. Yang, T. Kabasawa, T. Kato, M. Osakabe, H. Ohtake, A. Ishida, J. Lu, L. Zhang, N. Ohta, S. Kakehata, K. Joh, Q. Shi, X. Jin, J. Geng and M. Yamakawa (2016). “Thymic stromal lymphopoietin in tonsillar follicular dendritic cells correlates with elevated serum immunoglobulin A titer by promoting tonsillar immunoglobulin A class switching in immunoglobulin A nephropathy.” Transl Res 176: 1-17.
Nazli, A., J. K. Kafka, V. H. Ferreira, V. Anipindi, K. Mueller, B. J. Osborne, S. Dizzell, S. Chauvin, M. F. Mian, M. Ouellet, M. J. Tremblay, K. L. Mossman, A. A. Ashkar, C. Kovacs, D. M. Bowdish, D. P. Snider, R. Kaul and C. Kaushic (2013). “HIV-1 gp120 induces TLR2- and TLR4-mediated innate immune activation in human female genital epithelium.” J Immunol 191(8): 4246-4258.
Qin, T., Y. Yin, X. Wang, H. Liu, J. Lin, Q. Yu and Q. Yang (2015). “Whole inactivated avian Influenza H9N2 viruses induce nasal submucosal dendritic cells to sample luminal viruses via transepithelial dendrites and trigger subsequent DC maturation.” Vaccine 33(11): 1382-1392.
Rimoldi, M., M. Chieppa, V. Salucci, F. Avogadri, A. Sonzogni, G. M. Sampietro, A. Nespoli, G. Viale, P. Allavena and M. Rescigno (2005). “Intestinal immune homeostasis is regulated by the crosstalk between epithelial cells and dendritic cells.” Nat Immunol 6(5): 507-514.
Saghazadeh, A. and N. Rezaei (2017). “Implications of Toll-like receptors in Ebola infection.” Expert Opin Ther Targets 21(4): 415-425.
Sallusto, F. and A. Lanzavecchia (1994). “Efficient presentation of soluble antigen by cultured human dendritic cells is maintained by granulocyte/macrophage colony-stimulating factor plus interleukin 4 and downregulated by tumor necrosis factor alpha.” J Exp Med 179(4): 1109-1118.
Schmittgen, T. D. and K. J. Livak (2008). “Analyzing real-time PCR data by the comparative C(T) method.” Nat Protoc 3(6): 1101-1108.
Schon-Hegrad, M. A., J. Oliver, P. G. McMenamin and P. G. Holt (1991). “Studies on the density, distribution, and surface phenotype of intraepithelial class II major histocompatibility complex antigen (Ia)-bearing dendritic cells (DC) in the conducting airways.” J Exp Med 173(6): 1345-1356.
Smelter, D., M. Thompson, L. Meuchel, E. Townsend, A. Ryu, C. Pabelick, R. Vassallo and Y. S. Prakash (2009). “Thymic Stromal Lymphopoietin (TSLP) and Airway Smooth Muscle.” The FASEB Journal 23(1 supplement): 622.626-622.626.
Sripada, L., K. Singh, A. V. Lipatova, A. Singh, P. Prajapati, D. Tomar, K. Bhatelia, M. Roy, R. Singh, M. M. Godbole, P. M. Chumakov and R. Singh (2017). “hsa-miR-4485 regulates mitochondrial functions and inhibits the tumorigenicity of breast cancer cells.” J Mol Med (Berl) 95(6): 641-651.
Sun, J. C., S. Ugolini and E. Vivier (2014). “Immunological memory within the innate immune system.” Embo j 33(12): 1295-1303.
Takai, T. (2012). “TSLP Expression: Cellular Sources, Triggers, and Regulatory Mechanisms.” All ergology International 61(1): 3-17.
Takano, K., T. Kojima, M. Go, M. Murata, S. Ichimiya, T. Himi and N. Sawada (2005). “HLA-DR- and CD11c-positive dendritic cells penetrate beyond well-developed epithelial tight junctions in human nasal mucosa of allergic rhinitis.” J Histochem Cytochem 53(5): 611-619.
Tsilingiri, K., G. Fornasa and M. Rescigno (2017). “Thymic Stromal Lymphopoietin: To Cut a Long Story Short.” Cellular and Molecular Gastroenterology and Hepatology 3(2): 174-182.
Van Roey, G. A., M. A. Arias, J. S. Tregoning, G. Rowe and R. J. Shattock (2012). “Thymic stromal lymphopoietin (TSLP) acts as a potent mucosal adjuvant for HIV-1 gp140 vaccination in mice.” Eur J Immunol 42(2): 353-363.
Vliagoftis, H., A. Schwingshackl, C. D. Milne, M. Duszyk, M. D. Hollenberg, J. L. Wallace, A. D. Befus and R. Moqbel (2000). “Proteinase-activated receptor-2-mediated matrix metalloproteinase-9 release from airway epithelial cells.” J Allergy Clin Immunol 106(3): 537-545.
Wang, Q., J. Du, J. Zhu, X. Yang and B. Zhou (2015). “Thymic stromal lymphopoietin signaling in CD4(+) T cells is required for TH2 memory.” J Allergy Clin Immunol 135(3): 781-791 e783.
Wijkhuisen, A., A. Savatier, N. Cordeiro and M. Leonetti (2016). “Production of antigen-specific human IgGs by in vitro immunization.” BMC Biotechnol 16: 22.
Wilson, S. R., L. The, L. M. Batia, K. Beattie, G. E. Katibah, S. P. McClain, M. Pellegrino, D. M. Estandian and D. M. Bautista (2013). “The epithelial cell-derived atopic dermatitis cytokine TSLP activates neurons to induce itch.” Cell 155(2): 285-295.
Wygrecka, M., B. K. Dahal, D. Kosanovic, F. Petersen, B. Taborski, S. von Gerlach, M. Didiasova, D. Zakrzewicz, K. T. Preissner, R. T. Schermuly and P. Markart (2013). “Mast cells and fibroblasts work in concert to aggravate pulmonary fibrosis: role of transmembrane SCF and the PAR-2/PKC-alpha/Raf-1/p44/42 signaling pathway.” Am J Pathol 182(6): 2094-2108.
Yeh, C. Y., T. H. Yeh, C. J. Jung, P. L. Chen, H. T. Lien and J. S. Chia (2013). “Activated human nasal epithelial cells modulate specific antibody response against bacterial or viral antigens.” PLoS One 8(2): e55472.
Zaman, M., S. Chandrudu and I. Toth (2013). “Strategies for intranasal delivery of vaccines.” Drug Deliv Transl Res 3(1): 100-109.
Zhou, Z., L. Xu, A. Sennepin, C. Federici, Y. Ganor, D. Tudor, D. Damotte, N. Barry Delongchamps, M. Zerbib and M. Bomsel (2018). “The HIV-1 viral synapse signals human foreskin keratinocytes to secrete thymic stromal lymphopoietin facilitating HIV-1 foreskin entry.” Mucosal Immunol 11(1): 158-171.
Ziegler, S. F. (2012). “Thymic stromal lymphopoietin (TSLP) and allergic disease.” The Journal of allergy and clinical immunology 130(4): 845-852.

1. An immunoadjuvant composition comprising the P1 peptide of the HIV-1 envelope subunit gp41.

2. A method of improving the innate immunity of a subject in need thereof, comprising administering to the subject a therapeutically effective amount of the immunoadjuvant composition of claim 1, or a nucleic acid encoding the P1 peptide of the HIV-1 envelope subunit gp41.

3. (canceled)

4. A method of treating an infectious disease in a subject in need thereof comprising administering to the subject i) P1 peptide of the HIV-1 envelope subunit gp41 as an adjuvant, and ii) a treatment for the infectious disease.

5. The method of claim 4, wherein the P1 peptide of the HIV-1 envelope subunit gp41 and the treatment against the infectious diseases are administered as a combined preparation for simultaneous, separate or sequential use.

6. The according to claim 4, wherein the infectious disease is caused by a pathogen which is a virus, a bacteria or a parasite.

7. The immunoadjuvant composition according to claim 1 wherein the peptide P1 has a sequence set forth as in SEQ ID NO: 1.

8. The immunoadjuvant composition according to claim 1, wherein the peptide P1 has a sequence set forth as in SEQ ID NO: 2, 3 or 4.

9. (canceled)

10. A vaccine composition, comprising at least one antigen and at least the P1 peptide of the HIV-1 envelope subunit gp41 and, optionally, one or more pharmaceutically acceptable excipients.

11. A method of improving the innate immunity of a subject in need thereof comprising administering to the subject a therapeutically effective amount of the vaccine composition of claim 10.

12. A method of treating an infectious disease in a subject in need thereof comprising administering to the subject a therapeutically effective amount of the vaccine composition of claim 10.

13. (canceled)
